(19) United States
(12) Patent Application Publication
(10) Pub. No.: US 2022/0330873 A1
(43) Pub. Date: Oct. 20, 2022

(54) ELECTROCARDIOGRAPHIC MEASUREMENT DEVICE

(71) Applicant: OMRON HEALTHCARE Co., Ltd., Kyoto (JP)

(72) Inventors: Mika EZOE, Kyoto (JP); Mitsuru SAMEJIMA, Kyoto (JP); Shinya KODAKA, Kyoto (JP)

(21) Appl. No.: 17/662,776

(22) Filed: May 10, 2022

Related U.S. Application Data
(63) Continuation of application No. PCT/JP2020/041467, filed on Nov. 6, 2020.

(30) Foreign Application Priority Data
Nov. 15, 2019 (JP) 2019-207138

(51) Int. Cl. A61B 5/339; A61B 5/332; A61B 5/28

(52) U.S. Cl. CPC A61B 5/339; A61B 5/332; A61B 5/28; A61B 5/024

(57) ABSTRACT

An electrocardiographic measurement device according to the present invention includes a sensor capable of measuring an electrocardiographic waveform, an LED display unit, and a control means that executes a measurement process of the electrocardiographic waveform by controlling the sensor and controls the LED display unit to blink during the measurement process, wherein when the control means causes the LED display unit to blink during measurement of the electrocardiographic waveform by the sensor, the control means performs control such that luminance of the LED display unit changes with a predetermined gradient or more with respect to time.

### CROSS-REFERENCE TO RELATED APPLICATIONS

This application is the U.S. national stage application filed pursuant to 35 U.S.C. 365(c) and 120 as a continuation of International Patent Application No. PCT/JP2020/041467, filed Nov. 6, 2020, which application claims priority to Japanese Patent Application No. 2019-207138, filed Nov. 15, 2019, which applications are incorporated herein by reference in their entireties.

### TECHNICAL FIELD

The present invention belongs to the technical field related to healthcare, and particularly relates to an electrocardiographic measurement device.

### BACKGROUND ART

In recent years, it has become widespread to perform health management by measuring information (hereinafter, also referred to as biological information) on the body and health of an individual such as a blood pressure value and an electrocardiographic waveform with a measurement device, and by recording and analyzing the measurement result with an information terminal.

As an example of a measurement device as described above, a portable electrocardiographic measurement device configured to measure an electrocardiographic waveform immediately when anomaly occurs in everyday life, such as pain and palpitation in a chest, has been proposed, and an early detection of heart disease or a contribution to appropriate treatment is expected (for example, Patent Documents 1 and 2; Citations: Patent Document 1: JP 2005-000420 A and, Patent Document 2: JP 2007-105316 A).

### SUMMARY OF INVENTION

### Technical Problem

Patent Document 1 describes a portable electrocardiographic measurement device that includes a sensor unit, a control unit, an input unit, a display unit, a timer unit, and performs measurement of an electrocardiographic waveform, display during measurement, display of an analysis result, storage of the result, etc. in the same main body. On the other hand, Patent Document 2 describes a case where a display unit including an LED is provided in a main body of such a portable biological information measurement device, and where state information during measurement or the measured biological information is indicated by turning on or blinking of the LED. By displaying information in such a manner, it is possible to reduce the size and simplify the configuration related to the display unit of the portable device, and to improve the convenience of the portable device.

However, since the psychological stability of the user at the time of measurement is important for accurate electrocardiographic measurement, there is a problem that when such a method is used in an electrocardiographic measurement device, there is a possibility that the turning-on and blinking operation of the LED may adversely affect the psychology of the user, such as causing potential uneasiness, and accurate electrocardiograph measurement may be affected.

In view of the above-described conventional techniques, an object of the present invention is to provide a technique capable of accurately performing electrocardiographic measurement while suppressing a sense of uneasiness given to a user.

### Solution to Problem

To solve the above problem, the electrocardiographic measurement device according to the present invention is an electrocardiographic measurement device including a sensor capable of measuring an electrocardiographic waveform, an LED display unit, and a control means that executes a measurement process of the electrocardiographic waveform by controlling the sensor and controls the LED display unit to blink during the measurement process, wherein when the control means causes the LED display unit to blink during measurement of the electrocardiographic waveform by the sensor, the control means performs control such that luminance of the LED display unit changes with a predetermined gradient or more with respect to time.

According to such a configuration, in a case where the user is notified by blinking of the LED during the electrocardiographic measurement, the LED can be blinked with a gentle change in luminance. Therefore, it is possible to reduce a psychological load on the user and to suppress an adverse effect on the electrocardiographic measurement.

Further, change in the luminance of the LED display unit with the predetermined gradient or more with respect to time may be a shape of a triangular wave or a semicircular wave.

Further, the control means may apply a voltage to the LED display unit at a cycle synchronized with a heart rate of a measurement target. According to such a configuration, the user who performs measurement can be expected to be in a more psychologically stable state by the LED which blinks at a rhythm synchronized with his/her heart rate.

Further, the control means may apply the voltage to the LED display unit at a cycle of 40 to 60 times per minute. Further, the control means may apply the voltage to the LED display unit such that a turn-on time of the LED display unit is longer than a turn-off time thereof.

Further, the electrocardiographic measurement device may be a portable electrocardiographic measurement device.

### Advantageous Effects of Invention

According to the present invention, it is possible to provide an electrocardiographic measurement device capable of accurately performing electrocardiographic measurement while suppressing a sense of uneasiness given to a user.

### BRIEF DESCRIPTION OF DRAWINGS

FIG. 1 is a six-view diagram illustrating a configuration of a portable electrocardiographic measurement device according to an embodiment. (A) of FIG. 1 is a front view illustrating the configuration of the portable electrocardiographic measurement device according to the embodiment. (B) of FIG. 1 is a rear view illustrating the configuration of the portable electrocardiographic measurement device according to the embodiment. (C) of FIG. 1 is a left side view illustrating the configuration of the portable electrocardiographic measurement device according to the embodiment. (D) of FIG. 1 is a right side view illustrating the configuration of the portable electrocardiographic measurement device according to the embodiment. (E) of FIG. 1 is a plan view illustrating the configuration of the portable electrocardiographic measurement device according to the embodiment. (F) of FIG. 1 is a bottom view illustrating the configuration of the portable electrocardiographic measurement device according to the embodiment.

FIG. 2 is a block diagram illustrating a functional configuration of the portable electrocardiographic measurement device according to the embodiment.

FIG. 3 is a flowchart illustrating a flow of electrocardiographic waveform measurement processing in the portable electrocardiographic measurement device according to the embodiment.

FIG. 4(A) is a diagram illustrating a first waveform of an LED lighting pattern indicating a measurement state in the portable electrocardiographic measurement device according to the embodiment.

FIG. 4(B) is a diagram illustrating a second waveform of an LED lighting pattern indicating a measurement state in the portable electrocardiographic measurement device according to the embodiment.

FIG. 4(C) is a diagram illustrating a third waveform of an LED lighting pattern indicating a measurement state in the portable electrocardiographic measurement device according to the embodiment.

FIG. 4(D) is a diagram illustrating a fourth waveform of an LED lighting pattern indicating a measurement state in the portable electrocardiographic measurement device according to the embodiment.

### DESCRIPTION OF EMBODIMENTS

### First Embodiment

Embodiments of the present invention will be specifically described below with reference to the drawings. It should be noted that the dimension, material, shape, relative arrangement and the like of the components described in the present embodiment are not intended to limit the scope of this invention to them alone, unless otherwise stated.

### Electrocardiographic Measurement Device

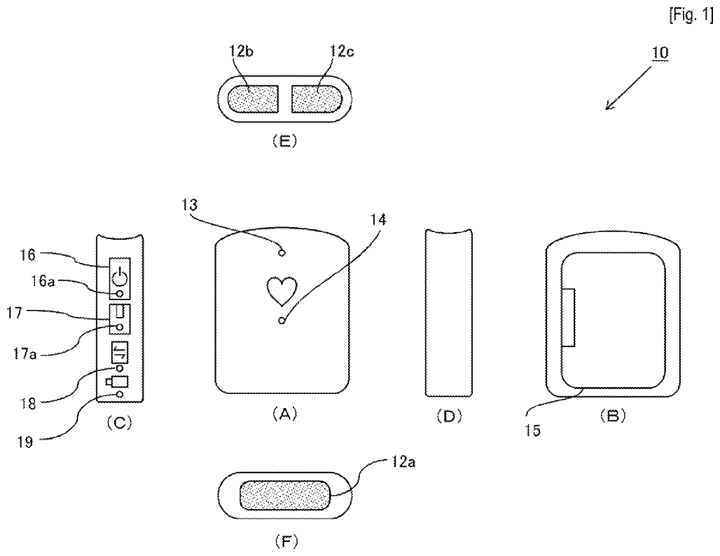

FIG. 1 is a diagram illustrating a configuration of a portable electrocardiograph 10 according to the present embodiment. (A) of FIG. 1 is a front view illustrating a front surface of the main body, and similarly, (B) of FIG. 1 is a rear view, (C) of FIG. 1 is a left side view, (D) of FIG. 1 is a right side view, (E) of FIG. 1 is a plan view, and (F) of FIG. 1 is a bottom view.

On the bottom surface of the portable electrocardiograph 10, a left electrode 12a to be brought into contact with the left side of the body at the time of electrocardiographic measurement is provided. On the upper surface side of the opposite side surface, a first right electrode 12b to be brought into contact with the pad of the index finger of the right hand and a second right electrode 12c to be brought into contact with the middle node of the index finger of the right hand are similarly provided. Note that the first right electrode 12b is an electrode that functions as a GND electrode.

At the time of electrocardiographic measurement, the portable electrocardiograph 10 is held by the right hand, and the index finger of the right hand is placed at the upper surface portion of the portable electrocardiograph 10 so as to correctly contact the first right electrode 12b and the second right electrode 12c. The left electrode is then brought into contact with one of the skins corresponding to the desired measurement. For example, when measurement is performed by the so-called I lead, the left electrode is brought into contact with the palm of the left hand, and when measurement is performed by the so-called V4 lead, the left electrode is brought into contact with the skin slightly to the left of the epigastric region of the left chest and below the papilla.

In addition, various types of operation units and indicators are arranged at the left side surface of the portable electrocardiograph 10. Specifically, a power switch 16, a power source LED 16a, a Bluetooth (registered trademark) Low Energy (BLE) communication button 17, a BLE communication LED 17a, a memory residual display LED 18, a battery exchange LED 19, and the like, are provided.

Additionally, a measurement state notification LED 13, an analysis result notification LED 14, and the like are provided at the front surface of the portable electrocardiograph 10, and a battery housing opening and a battery cover 15 are arranged at the rear surface of the portable electrocardiograph 10.

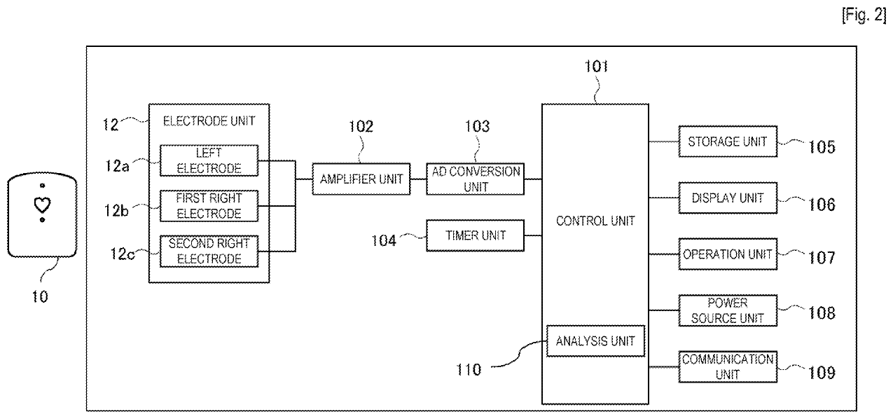

Also, in FIG. 2, a block diagram illustrating a functional configuration of the portable electrocardiograph 10 is described. As illustrated in FIG. 2, the portable electrocardiograph 10 includes each functional unit of a control unit 101, such as an electrode unit 12, an amplifier unit 102, an AD (Analog to Digital) conversion unit 103, a timer unit 104, a storage unit 105, a display unit 106, an operation unit 107, a power source unit 108, a communication unit 109, and an analysis unit 110.

The control unit 101 is a means for controlling the portable electrocardiograph 10, and includes, for example, a central processing unit (CPU). Upon receiving the operation of the user via the operation unit 107, the control unit 101 controls each component of the portable electrocardiograph 10 to execute various processes such as electrocardiographic measurement, information communication, and the like according to a predetermined program. The predetermined program is stored in the storage unit 105, which will be described later, and is read therefrom.

The control unit 101 includes the analysis unit 110 configured to analyze the electrocardiographic waveform as a function module. The analysis unit 110 analyzes the presence or absence of the disturbance of the waveform and the like for the measured electrocardiographic waveform, and outputs at least a result indicating whether the electrocardiographic waveform at the time of measurement is normal.

The electrode unit 12 includes the left electrode 12a, the first right electrode 12b, and the second right electrode 12c, and functions as a sensor for detecting an electrocardiographic waveform. The amplifier unit 102 has a function of amplifying the signal output from the electrode unit 12. The AD conversion unit 103 converts the analog signal amplified by the amplifier 102 into a digital signal, and has a function to transmit it to the control unit 101.

The timer unit 104 has a function of measuring time with reference to the RTC (Real Time Clock). As will be described later, for example, the time until the end of measurement is counted at the time of electrocardiographic measurement, and is output.

The storage unit 105 includes a main storage device such as a random access memory (RAM), and stores various kinds of information such as an application program, a measured electrocardiographic waveform, and an analysis result. In addition to the RAM, for example, a long term storage medium such as flash memory may be provided.

The display unit 106 is configured to include the power source LED 16a, the BLE communication LED 17a, the memory residual display LED 18, the battery exchange LED 19, and the like described above, and transmits the state of the device to the user by turning on or blinking the LED. Furthermore, the operation unit 107 includes the power switch 16, the communication button 17, and the like, and receives input operation from a user, and has a function for causing the control unit 101 to execute a process in response to the operation.

The power source unit 108 is configured to include a battery that supplies the power required for operation of the device. The battery may be a secondary battery such as a lithium ion battery, for example, or may be a primary battery.

The communication unit 109 includes an antenna for wireless communication, and has a function of communicating with another device such as an information processing terminal by at least BLE communication. Alternatively, a terminal for communication by wired line may be provided.

### Electrocardiographic Measurement Process Using Portable Electrocardiograph

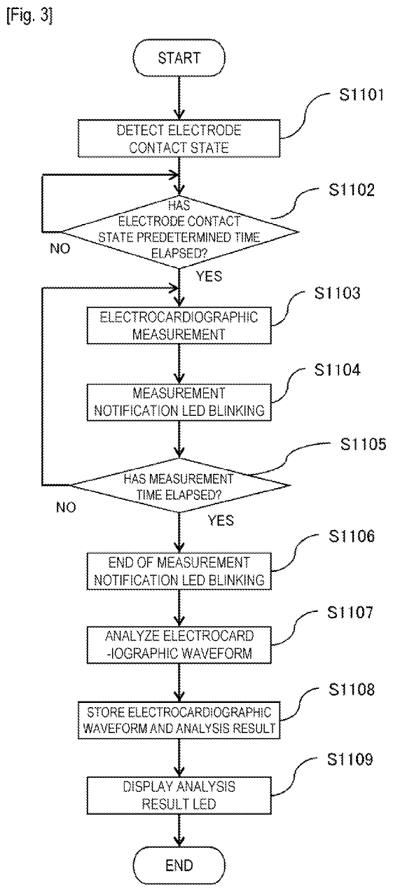

Next, the operation of the portable electrocardiograph 10 when performing the electrocardiographic measurement is described on the basis of FIGS. 1, 2, and 3. FIG. 3 is a flowchart illustrating a procedure of processing when performing electrocardiographic measurement using the portable electrocardiograph 10.

Prior to measurement, the user operates the power switch 16 to turn ON the power source of the portable electrocardiograph 10. As a result, the power source LED is turned ON to indicate that the power source is ON. Then, the portable electrocardiograph 10 is held in the right hand, the index finger of the right hand is brought into contact with the 12b and the 12c, and the 12a is brought into contact with a portion of the skin to be measured. Then, the control unit 101 detects a contact state via the electrode unit 12 (S1101), and executes a process of determining whether a predetermined time has elapsed with the electrode correctly in contact (S1102). Here, if it is determined that the predetermined time has not elapsed, the control unit 101 repeats the same processing until the predetermined time elapses, and if it is determined that the predetermined time has elapsed, the process proceeds to step S1103, and actual electrocardiographic measurement is performed.

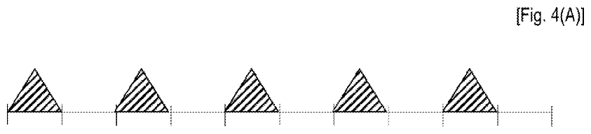

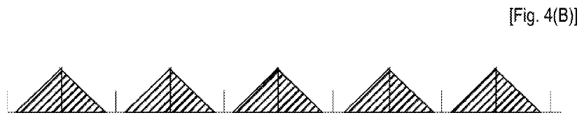

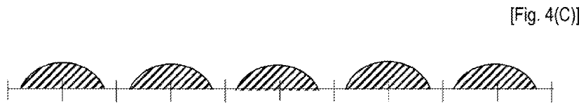

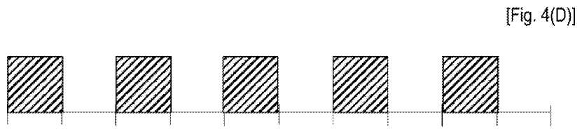

While the electrocardiographic measurement is performed, the control unit 101 stores the measurement value in the storage unit 105 at any time, and displays that the electrocardiographic measurement is being performed by blinking the measurement state notification LED 13 on the front surface of the main body at a predetermined rhythm (S1104). Here, the blinking of the measurement state notification LED 13 will be described in detail with reference to FIGS. 4(A)-4(D). Each waveform illustrated in FIGS. 4(A)-4(D) illustrates an aspect of applying a voltage to the measurement state notification LED 13.

In general, psychological stability of the user at the time of measurement is important for accurate electrocardiographic measurement, and for example, when a voltage is applied to the LED with a waveform as illustrated in FIG. 4(D) to cause the LED to blink, the LED is rapidly switched between turn-on and turn-off, resulting in a flickering blinking operation. Such a blinking manner of the warning lamp may cause a sense of uneasiness and tension of the user, and when the measurement is performed in such a psychological state, accurate electrocardiographic measurement may not be performed.

Therefore, in the portable electrocardiograph 10 according to the present embodiment, the control unit 101 performs control so that the luminance of the measurement state notification LED 13 changes with a predetermined gradient or more with respect to time as illustrated as a triangular waveform as illustrated in FIG. 4(A) and FIG. 4(B) or a semicircular waveform as illustrated in FIG. 4(C), thereby causing the LED to blink gently. Further, as illustrated in FIG. 4(A), FIG. 4(B), and FIG. 4(C), the control unit 101 performs control such that the measurement state notification LED 13 blinks so that the turn-on time is longer than the turn-off time. Accordingly, even when the user sees the blinking of the LED notifying that the electrocardiographic measurement is being performed, the user is less likely to feel uneasy, and the user can contribute to accurate electrocardiographic measurement. In addition, blinking may be performed in a cycle synchronized with the heart rate of the user at the time of measurement.

Returning to the description of the flow of the electrocardiographic measurement process, in step S1105, the control unit 101 executes a process of determining whether the time of the electrocardiographic measurement has exceeded a predetermined measurement time (for example, 30 seconds). Here, if it is determined that the predetermined amount of time has not elapsed, the process returns to step S1103, and the subsequent processing is repeated. On the other hand, if it is determined that the predetermined measurement time has elapsed, the measurement is completed, and a process of terminating the blink of the measurement state notification LED 13 is executed (step S1106).

Next, the analysis unit 110 of the control unit 101 performs analysis of the measured data (electrocardiographic waveform) stored in the storage unit 105 (S1107), and the analysis result is stored in a long term storage device along with the electrocardiographic waveform (S1108). Then, the control unit 101 displays the result of the analysis by the analysis result notification LED 14 (S1109), and ends the series of processes. Note that the analysis result may be displayed, for example, by turning on the LED only when there is an anomaly in the electrocardiographic waveform, or by turning on and blinking the LED according to the analysis result.

According to the portable electrocardiograph 10 of the present embodiment having the above-described configuration, since the LED indicating that the electrocardiograph is being measured blinks with a gentle change in luminance, it is possible to suppress a sense of uneasiness given to a user.

### Other Points

The description of the embodiment described above is merely illustrative of the present invention, and the present invention is not limited to the specific embodiments described above. Within the scope of the technical idea of the present invention, various modifications and combinations may be made.

For example, various notifications using turning on or blinking of an LED may indicate the notification content by changing the display color of the LED. According to such a configuration, it is also possible to diversify notification contents.

In addition, although the portable electrocardiograph according to the above-described embodiment is configured to include the BLE communication function, the communication function is not an essential configuration, and an electrocardiograph without the communication function may be configured.

### REFERENCE NUMERALS LIST

10 Portable electrocardiograph
13 Measurement state notification LED
14 Analysis result notification LED
15 Battery cover
16 Power switch
16a Power source LED
17 Communication button
17a BLE communication LED
18 Memory residual display LED
19 Battery exchange LED

1. An electrocardiographic measurement device comprising:
a sensor capable of measuring an electrocardiographic waveform;
an LED display unit; and
a control means that executes a measurement process of the electrocardiographic waveform by controlling the sensor and controls the LED display unit to blink during the measurement process, wherein
when the control means causes the LED display unit to blink during measurement of the electrocardiographic waveform by the sensor, the control means performs control such that luminance of the LED display unit changes with a predetermined gradient or more with respect to time, and
change in the luminance of the LED display unit with the predetermined gradient or more with respect to time is a shape of a triangular wave or a semicircular wave.

2. The electrocardiographic measurement device according to claim 1, wherein
the control means causes the LED display unit to blink at a cycle synchronized with a heart rate of a measurement target.

3. The electrocardiographic measurement device according to claim 1, wherein
the control means causes the LED display unit to blink at a cycle of 40 to 60 times per minute.

4. An electrocardiographic measurement device comprising:
a sensor capable of measuring an electrocardiographic waveform;
an LED display unit; and
a control means that executes a measurement process of the electrocardiographic waveform by controlling the sensor and controls the LED display unit to blink during the measurement process, wherein
when the control means causes the LED display unit to blink during measurement of the electrocardiographic waveform by the sensor, the control means causes the LED display unit to blink such that luminance of the LED display unit changes with a predetermined gradient or more with respect to time and a turn-on time of the LED display unit is longer than a turn-off time thereof.

5. The electrocardiographic measurement device according to claim 1, wherein
the electrocardiographic measurement device is a portable electrocardiographic measurement device.

6. The electrocardiographic measurement device according to claim 2, wherein
the control means causes the LED display unit to blink at a cycle of 40 to 60 times per minute.

7. The electrocardiographic measurement device according to claim 4, wherein
the electrocardiographic measurement device is a portable electrocardiographic measurement device.
